(19) United States
(12) Patent Application Publication
(10) Pub. No.: US 2013/0160353 A1
(43) Pub. Date: Jun. 27, 2013

(54) Biological Oils for Use in Compression Engines and Methods for Producing Such Oils

(71) Applicant: BP Corporation North America Inc., Naperville, IL (US)

(72) Inventors: Ryan Shea, Naperville, IL (US); Jon Hanson, West Friendship, MD (US); Kirk E. Apt, Ellicott City, MD (US); Bill Barclay, Columbia, MD (US); Jacob Borden, San Diego, CA (US); Michael Foster, Downers Grove, IL (US)

(73) Assignee: BP Corporation North America Inc., Naperville, IL (US)

(21) Appl. No.: 13/727,103

(22) Filed: Dec. 26, 2012

Related U.S. Application Data
(60) Provisional application No. 61/580,450, filed on Dec. 27, 2011.

(51) Int. Cl. C10L 1/18; C07C 69/003; C12P 7/64

(52) U.S. Cl. CPC C10L 1/1802; C12P 7/6463; C07C 69/003; C12P 7/649
USPC 44/308; 554/1; 554/227; 44/388; 435/134; 44/307; 435/289.1

(57) ABSTRACT

Disclosed herein are oils, including fatty acid triglyceride and fatty acid ester compositions suitable for the production of biofuel, biofuels, and methods for producing such materials and compositions. These materials and compositions comprise substantially no sterol glycosides.

This application claims the benefit of U.S. Provisional Patent Application 61/580,450 filed on Dec. 27, 2011, which is incorporated by reference herein in its entirety.

### NAMES OF THE PARTIES TO A JOINT RESEARCH AGREEMENT

For purposes of 35 U.S.C. §103(c)(2), a joint research agreement was executed between BP Biofuels UK Limited and Martek Biosciences Corporation on Dec. 18, 2008 in the field of biofuels. Also for the purposes of 35 U.S.C. §103(c)(2), a joint development agreement was executed between BP Biofuels UK Limited and Martek Biosciences Corporation on Aug. 7, 2009 in the field of biofuels. Also for the purposes of 35 U.S.C. §103(c)(2), a joint development agreement was executed between BP Biofuels UK Limited and DSM Biobased Products and Services B.V. on Sep. 1, 2012 in the field of biofuels.

### BACKGROUND

1. Technical Field

The invention is directed to biological oils and biofuels suitable for use in compression engines, and to methods, units, and microorganisms for producing same.

2. Discussion of Related Art

Issues of greenhouse gas levels and climate change have led to development of technologies seeking to utilize natural cycles between fixed carbon and liberated carbon dioxide. As these technologies advance, various techniques to convert feedstocks into biofuels have been developed. However, even with the above advances in technology, there remains a need and a desire to improve economic viability for conversion of renewable carbon sources to fuels.

Biofuels such as biodiesel can be produced from vegetable oils by reactions such as transesterification to convert fatty acid triglycerides into fatty acid methyl esters (FAME).

Sterol glycosides (SGs), in particular sterol glucosides, occur naturally in vegetable oils and fats in the acylated form. An example of a sterol glucoside in both the free and acylated form is shown below (stereochemistry eliminated for conveniences.

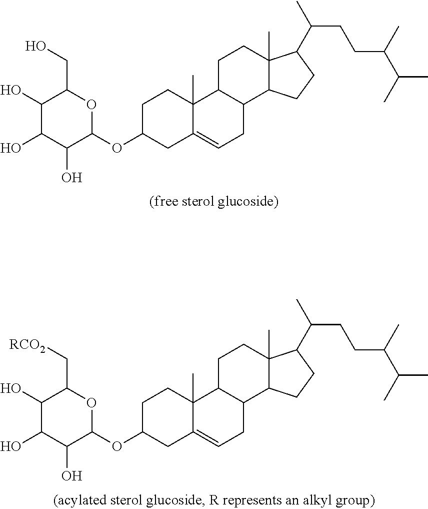

While acylated, sterol glycosides are very soluble in vegetable oil. However, during the biodiesel conversion process, they are typically converted to nonacylated SGs. The presence of SGs in biodiesel can contribute to flowability problems in biodiesel and biodiesel blends. Due to the high melting point of SGs and their insolubility in biodiesel or diesel fuel, these materials may negatively impact the cold-flow properties of the resulting fuel, e.g., cloud point, pour point, cold-filter plugging, etc. Amounts as low as 10-15 ppm by weight of SGs in biofuel are enough to cause these problems.

Thus, there is a need for biofuels that are substantially free of sterol glycosides.

### SUMMARY

It has been unexpectedly discovered that certain microorganisms produce oils that are substantially free of sterol glycosides, and in particular sterol glucosides. Thus, in one aspect, the invention provides oils suitable for the production of biofuel, wherein the oils comprise material obtained from a culture comprising one or more oleaginous microorganisms and substantially no sterol glycosides.

In another aspect, the invention provides biofuels comprising substantially no sterol glycosides, and at least one fatty acid C₁-C₄ alkyl ester derived from an oil obtained from a culture comprising one or more oleaginous microorganisms.

In still another aspect, the invention provides biofuels made from the oils of the invention. In certain aspects, these biofuels meet or exceed the biodiesel standard set forth in ASTM standard specification D6751-11b. In other aspects, these biofuels meet or exceed the standard set forth in European Standard EN 14214.

In another aspect, the invention provides methods for producing a feedstock suitable for use in the production of a biofuel. These methods comprise growing a culture of at least one oleaginous microorganism in a presence of a carbon source, wherein material produced by the culture comprises substantially no sterol glycosides.

In another aspect, the invention provides biofuels produced by the methods of the invention.

Still further, the invention provides methods for producing a fatty acid derivative composition. These methods comprise growing a culture of at least one oleaginous microorganism in the presence of a carbon source, wherein material produced by the microorganism comprises substantially no sterol glycosides. In certain embodiments, these methods further comprise lysing the microorganism to produce a lysate. These methods may further comprise isolating an oil from the lysate.

In another aspect, the invention provides fatty acid methyl ester compositions comprising substantially no sterol glycosides, and at least one fatty acid methyl ester derived from an oil obtained from a culture comprising one or more oleaginous microorganisms.

In yet another aspect, the invention provides a fatty acid triglyceride composition comprising substantially no sterol glycosides, and at least one fatty acid triglyceride obtained from a culture comprising one or more oleaginous microorganisms.

Yet further, the invention provides production systems comprising a fatty triglyceride production vessel adapted to receive a carbon source, and a culture of an oleaginous microorganism, wherein material produced by the microorganism comprises substantially no sterol glycosides.

The invention also provides compositions produced by a microorganism, where the composition comprises substantially no sterol glycosides and one or more fatty acid derivatives.

The invention further provides methods for producing sterol glycoside free compositions that involve no processing to reduce sterol glycosides levels.

The invention also provides engines operating on a biofuel made from the oils, fatty acid triglyceride compositions, or fatty acid alkyl ester compositions of the invention.

In another aspect the invention includes a biofuel meeting or exceeding one or more of the following:
biodiesel standard set forth in ASTM standard specification D6751-11b or the European specification EN14214;
low temperature flow test as set forth in ASTM standard specification D4539;
cold filter plugging point test as set forth in ASTM standard specification D6371 or in European test method EN116. cloud point as set forth in the ASTM standard specification D2500 or in European test method EN23015.; and/or
pour point as set forth in the ASTM standard specification D6751 or in European test method EN3016.

### BRIEF DESCRIPTION OF THE DRAWINGS

The accompanying drawings, which are incorporated in and constitute a part of this specification, illustrate embodiments of the invention and, together with the description, serve to explain the features, advantages, and principles of the invention.

FIG. 1 is a chromatogram (GC-MS) for the material produced in Example 1.

FIG. 2 is a chromatogram (GC-MS) for the material produced in Example 2.

FIG. 3 is a schematic representation of a unit, according to some embodiments.

FIG. 4 is a schematic representation of a two stage unit, according to some embodiments.

FIG. 5 is a schematic representation of a unit with extraction, according to some embodiments.

FIG. 6 is a schematic representation of a fermentor, according to some embodiments.

FIG. 7. Is a chromatogram (GC-MS) of a standard (Matraya) containing sterol glycosides.

### DETAILED DESCRIPTION

The invention provides oils and biofuels suitable for use in compression engines and also provides methods, systems, units, and/or organisms suitable for use in preparing or manufacturing such oils and biofuels. The oils of the invention are obtained from microorganisms, in particular oleaginous microorganisms, and more particularly cultures of one or more oleaginous microorganisms. The oils of the invention may be used as biofuels or conveniently converted to biofuels, in particular biodiesel.

The invention also provides fatty acid triglyceride compositions and fatty acid ester compositions. These compositions may also be used directly as biofuels or may be conveniently converted to such products. These compositions are obtained from microorganisms, in particular oleaginous microorganisms, and more particularly cultures of one or more oleaginous microorganisms.

The oils, triglyceride compositions, ester compositions, and biofuels of the invention are substantially free of sterol glycosides, and, in particular, sterol glucosides. In some embodiments, the biofuels comprise less than about 15 parts per million of sterol glycosides by weight. In various embodiments, the oils, triglyceride compositions, and fatty acid ester compositions comprise less than about 15 parts per million of sterol glycosides by weight. In other embodiments, the oils, triglyceride compositions, and fatty acid ester compositions comprise an amount of sterol glycosides sufficient to yield a biofuel having no more than about 15 parts per million by weight of the glycosides after additional processing or blending of the oil, triglyceride composition, or fatty acid ester composition to yield the biofuel.

In some embodiments, the oils, triglyceride compositions, and fatty acid ester compositions comprise less than about 10 parts per million of sterol glycosides by weight. In other embodiments, the oils, triglyceride compositions, and fatty acid ester compositions comprise less than about 5 parts per million of sterol glycosides by weight.

In some embodiments of the invention, the amount of sterol glycosides in the oils, triglyceride compositions, and fatty acid ester compositions will be no greater than an amount sufficient to yield a biofuel that meets ASTM standard specification D6751-11b when the oil, triglyceride composition, or fatty acid ester composition is further processed or blended to produce a biofuel.

In various embodiments of the invention, the biofuels meet or exceed the biodiesel standard set forth in the ASTM standard specification D6751-11b.

The ASTM standard specification D6751-11b referred to herein was published in July, 2011. The content of ASTM standard specification D6751-11b is incorporated herein by reference in its entirety.

In some embodiments of the invention, the biofuel has a cold-soak filtration time of less than 360 seconds as determined using the procedure set forth in ASTM standard test method D7501-09b.

The ASTM standard specification 7501-09b referred to herein was published in November 2009. The content of the ASTM standard specification 7501-09b is incorporated herein in its entirety.

In some embodiments of the invention, the amount of sterol glycosides in the oils, triglyceride compositions, and fatty acid ester compositions will be no greater than an amount sufficient to yield a biofuel that meets European Standard specification EN14214:2003 when the oil, triglyceride composition, or fatty acid ester composition is further processed or blended to produce a biofuel.

In various embodiments of the invention, the biofuels meet or exceed the standard set forth in the European Standard specification EN14214:2003.

The European Standard specification EN14214:2003 referred to herein was published in July, 2003. The content of European Standard specification EN14214:2003 is incorporated herein by reference in its entirety. In some embodiments, the biofuels of the invention comprise less than about 10 parts per million of sterol glycosides by weight. In other embodiments, the biofuels of the invention comprise less than about 5 parts per million of sterol glycosides by weight.

In some embodiments of the invention, the fatty acids in the oils, triglyceride compositions, ester compositions, and biofuels have a rapeseed-like fatty acid profile.

Suitable organisms for use in the invention include those having an ability to yield greater than about 25 percent, greater than about 50 percent, greater than about 75 percent, about 99 percent, about 45 percent to about 90 percent, and/or the like of dry weight of the organism as lipid. In an embodiment, suitable organisms include those that yield an equivalent and/or a better amount of biomass when grown on xylose, sucrose, and/or glycerol as compared to a yield on primarily glucose alone.

According to some embodiments, the organism can yield greater than about 25 percent, greater than about 50 percent, greater than about 75 percent, about 99 percent, about 25 percent to about 99 percent, and/or the like more fatty acids when grown on a combination of primarily sugar (e.g., sucrose, glucose, fructose, xylose, and/or the like) and glycerol as compared to primarily the sugar alone. The ratio of sugar to glycerol can be any suitable amount, such as about 100:1, about 50:1, about 10:1, about 1:1, about 1:10, about 1:50, about 1:100, about 1-20:50-100, and/or the like on, a mass basis, a mole basis, a volume basis, and/or the like.

According to some embodiments, the organism or culture of one or more oleaginous microorganisms comprises a bacteria, cyanobacteria, algae, or fungus.

According to certain embodiments, the organism or culture of one or more oleaginous microorganisms comprises a yeast.

According to some embodiments, the microorganisms and cultures include those of a genus of Rhodospondium, Pseudozyma, Tremelia, Rhodotorula, Sporidiobolus, Sporobolornyces, Ustilago, Cryptococcus, Leucosporidium, Candida, or combinations thereof. Other suitable microorganisms and cultures include those of a genus of Schizochytrium, Thraustochytrium, Ulkenia, Chlorella, Prototheca, or combinations thereof.

In some embodiments, the organism or culture of one or more oleaginous microorganisms comprises a member of the kingdom stramenopile, such as a thraustochytrid and/or golden algae, for example. The organism can be of the genus Schizochytrium, Thraustochytrium, Ulkenia, and/or the like.

In some embodiments, the organism or culture of one or more oleaginous microorganisms comprises a single cell member of the fungal kingdom, such as a yeast, for example. The organism can be of the genus Rhodosporidium, Leucosporidium, Pseudozyma, Tremella, Rhodotorula, Sporidiobolus, Sporobolomyces, Ustilago, Cryptococcus, Leucospondium, Candida, and/or the like.

In some embodiments, the organism or culture of one or more micro-organism comprises a basidiomycete yeast or an ascomycete yeast.

According to some embodiments, the organism or culture of one or more oleaginous microorganisms comprises Pseudozyrna aphidis, Pseudozyma sp., Rhodosporidium fluviale, Rhodosporidium paludigenum, Rhodotorula glutinis, Rhodotorula hordea, Rhodotorula ingenosa, Sporobolomyces ruberrimus, Trernella sp., Ustilago sp., Rhodosporidium toruloides including CBS 6016, Rhodosporidium toruloides including CBS 8587, Sporidiobolus pararoseus, Leucosporidium scottii, Pseudozyma antarctica, Rhodosporidium sphaerocarpum, Rhodotorula muscorum, Cryptococcus laurentii, Candida tropicalis, Rhodosporidium diobovatum, and/or the like.

In some embodiments, the organism or culture of one or more oleaginous microorganisms comprises a yeast selected from the genera of Rhodotorula and Sporidiobolus.

In other embodiments, the organism or culture of one or more oleaginous microorganisms is a yeast selected from the genera of Rhodotorula and Sporidiobolus.

According to some embodiments, the organism or culture of one or more oleaginous microorganisms comprises Rhodotorula ingenosa, Sporidiobolus pararoseus, or combinations thereof.

According to some embodiments, the organism or culture of one or more oleaginous microorganisms comprises Rhodotorula ingenosa. In other embodiments, the organism or culture of one or more oleaginous microorganisms is Rhodotorula ingenosa.

According to some embodiments, the organism or culture of one or more oleaginous microorganisms comprises Sporidiobolus pararoseus. According to other embodiments, the organism or culture of one or more oleaginous microorganisms is Sporidiobolus pararoseus.

According to some embodiments, the organism or culture of one or more oleaginous microorganisms comprises an organism having the identifying characteristics of ATCC Patent Deposit Designation of PTA-11616. In some embodiments, the culture contains a single microorganism which has the identifying characteristics of ATCC Patent Deposit Designation of PTA-11616.

According to some embodiments, the organism or culture of one or more oleaginous microorganisms comprises an organism having the identifying characteristics of ATCC Patent Deposit Designation of PTA-11617. In some embodiments, the culture contains a single microorganism which has the identifying characteristics of ATCC Patent Deposit Designation of PTA-11617.

In certain embodiments, the organism or culture of one or more oleaginous microorganisms comprises an eukaryotic microalgae.

In certain embodiments, the organism or culture of one or more oleaginous microorganisms comprises a Chlorella species.

In certain embodiments, the invention is directed to a fatty acid methyl ester composition comprising substantially no sterol glycosides, and at least one fatty acid methyl ester derived from an oil obtained from a culture comprising one or more oleaginous microorganisms. These organisms or cultures of one or more microorganisms include those described herein.

According to some embodiments, the invention is directed to a fatty acid triglyceride composition comprising substantially no sterol glycosides, and at least one fatty acid triglyceride obtained from a culture comprising one or more oleaginous microorganisms. The invention is further directed to a biofuel comprising this fatty acid methyl ester composition. The organisms or cultures of one or more microorganisms include those described herein.

According to some embodiments, the invention is directed to a biological oil containing fatty acids where the oil is made by any of the methods, units, and/or organisms disclosed within this specification.

The fatty acids in the oils, biofuels, triglycerides, and fatty acid ester compositions of the invention can have any suitable profile and/or characteristics, such as a profile generally suitable for biofuel production. According to some embodiments, the fatty acids can include a suitable amount and/or percent of fatty acids with four or more double bonds on a mass basis. In the alternative, the fatty acids can include a suitable amount and/or percent fatty acids with three or more double bonds, with two or more double bonds, with one or more double bonds, and/or the like.

The fatty acids in the oils, biofuels, triglycerides, and fatty acid ester compositions of the invention comprise from about 8 carbon atoms to about 30 carbon atoms.

The fatty acids in the oils, biofuels, triglycerides, and fatty acid ester compositions of the invention comprise from about 14 to about 24 carbon atoms.

According to some embodiments of the oils, biofuels, triglycerides, and fatty acid ester compositions of the invention, less than about 1 percent, of the fatty acids present in the material have four or more double bonds, based on the mass of total fatty acids.

According to some embodiments of the oils, biofuels, triglycerides, and fatty acid ester compositions of the invention, less than about 35 percent of the fatty acids present in the material are saturated fatty acids, based on the mass of total fatty acids.

The suitable amount of carbon-carbon double bonds can be less than about 25 percent as weight percent of total fatty acids, less than about 15 percent as weight percent of total fatty acids, less than about 10 percent as weight percent of total fatty acids, less than about 5 percent as weight percent of total fatty acids, less than about 3 percent as weight percent of total fatty acids, less than about 2 percent as weight percent of total fatty acids, less than about 1 percent as weight percent of total fatty acids, less than about 0.5 percent as weight percent of total fatty acids, less than about 0.1 percent as weight percent of total fatty acids, at least about 5 percent as weight percent of total fatty acids, about 25 percent as weight percent of total fatty acids to about 0.1 percent as weight percent of total fatty acids, about 10 percent as weight percent of total fatty acids to about 5 percent as weight percent of total fatty acids, and/or the like.

In addition and/or the alternative, the resulting fatty acids can include at least about 30 percent monounsaturated fatty acids as weight percent of total fatty acids, at least about 40 percent monounsaturated fatty acids as weight percent of total fatty acids, at least about 50 percent monounsaturated fatty acids as weight percent of total fatty acids, at least about 60 percent monounsaturated fatty acids as weight percent total fatty acids, at least about 70 percent monounsaturated fatty acids as weight percent of total fatty acids, at least about 80 percent monounsaturated fatty acids as weight percent of total fatty acids, at least about 90 percent monounsaturated fatty acids as weight percent of total fatty acids, about 30 percent monounsaturated fatty acids as weight percent of total fatty acids to about 90 percent monounsaturated fatty acids as weight percent of total fatty acids, less than about 100 percent monounsaturated fatty acids as weight percent of total fatty acids, about 50 percent monounsaturated fatty acids as weight percent of total fatty acids to about 70 percent monounsaturated fatty acids as weight percent of total fatty acids, and/or the like. Monounsaturated refers to molecules having one double bond.

According to some embodiments, the lipid can include any suitable amount and/or percent of saturated fatty acids on a total fatty acid mass basis. The suitable amount and/or percent of saturated fatty acids can include less than about 5 percent of total fatty acids as weight percent total fatty acids, less than about 10 percent of total fatty acids as weight percent total fatty acids, less than about 20 percent of total fatty acids as weight percent total fatty acids, less than about 25 percent of total fatty acids as weight percent total fatty acids, less than about 30 percent of total fatty acids as weight percent total fatty acids, less than about 35 percent of total fatty acids as weight percent total fatty acids, less than about 40 percent of total fatty acids as weight percent total fatty acids, less than about 50 percent of total fatty acids as weight percent total fatty acids, less than about 60 percent of total fatty acids as weight percent total fatty acids, at least about 1 percent of total fatty acids as weight percent total fatty acids, about 10 percent of total fatty acids as weight percent total fatty acids to about 60 percent of total fatty acids as weight percent total fatty acids, about 25 percent of total fatty acids as weight percent total fatty acids to about 40 percent of total fatty acids as weight percent total fatty acids, about 1 percent of total fatty acids as weight percent total fatty acids to about 10 percent of total fatty acids as weight percent total fatty acids and/or the like. Saturated refers to compounds with no double bonds and/or triple bonds between adjacent carbon atoms.

In some embodiments, the invention is directed to a biofuel suitable for use in a compression engine. In certain embodiments of the invention, the biofuel comprises a fatty acid methyl ester composition comprising a fatty acid methyl ester profile (expressed as weight percent of total fatty acids) having about 30 percent oleic acid to about 90 percent oleic acid, about 50 percent oleic acid to about 70 percent oleic acid, about 60 percent oleic acid, and/or the like. The profile can include about 10 percent linoleic acid to about 70 percent linoleic acid, about 30 percent linoleic acid to about 50 percent linoleic acid, about 15 percent linoleic acid to about 35 percent linoleic acid, about 40 percent linoleic acid, and/or the like.

According to some embodiments, the fatty acid methyl ester profile (expressed as weight percent of total fatty acids) can include: about 1 percent palmitic acid to about 10 percent palmitic acid; about 0.5 percent stearic acid to about 2.5 percent stearic acid; about 50 percent oleic acid to about 70 percent oleic acid; about 15 percent linoleic acid to about 35 percent linoleic acid; and/or about 6 percent linolenic acid to about 12 percent linolenic acid.

According to some embodiments, the fatty acid methyl ester profile (expressed as weight percent of total fatty acids) can include: about 0 percent myristic acid to about 1.5 percent myristic acid; about 1 percent palmitic acid to about 10 percent palmitic acid; about 0.5 percent stearic acid to about 2.5 percent stearic acid; about 0 percent arachidic acid to about 1.5 percent arachidic acid; about 0 percent behenic acid to about 1.5 percent behenic acid; about 0 percent lignoceric acid to about 2 percent lignoceric acid; about 0 percent palmitoleic acid to about 1 percent palmitoleic acid; about 50 percent oleic acid to about 70 percent oleic acid, about 0 percent eicosenoic acid to about 3 percent eicosenoic acid; about 0 percent erucic acid to about 5 percent erucic acid, about 15 percent linoleic acid to about 35 percent linoleic acid; and/or about 6 percent linolenic acid to about 12 percent linolenic acid.

According to some embodiments, the fatty acids can include a profile at least substantially similar to the fatty acids found in rapeseed. Substantially similar can include having a profile at least about 50 percent of rapeseed, at least about 60 percent of rapeseed, at least about 70 percent of rapeseed, at least about 80 percent of rapeseed, at least about 90 percent of rapeseed, at least about 95 percent of rapeseed, at least 99 percent of rapeseed, less than about 90 percent of rapeseed, about 50 percent of rapeseed to about 99 percent of rapeseed, and/or the like, all on a weight percent total fatty acid basis, as measured, for example, by correlation analysis, using, for example, r² values.

According to some embodiments, the invention is directed to methods of producing the oils, triglyceride compositions, fatty acid ester compositions, and biofuels of the invention. The methods include producing and/or growing an organism or culture of one or more oleaginous microorganisms. The methods employ the organisms or cultures of one or more microorganisms described herein. Producing and/or growing can be accomplished simultaneously and/or sequentially.

The oils, fatty acid triglyceride compositions, fatty acid alkyl ester compositions, and biofuels may be conveniently produced by methods that include consumption and conversion of sugar-containing carbon sources by the microorganisms or cultures of one or more oleaginous microorganisms described herein.

According to some embodiments, the microorganism or culture of one or more oleaginous microorganisms consumes a feedstock, where the feedstock includes sucrose, glucose, fructose, xylose, glycerol, mannose, arabinose, lactose, galactose, maltose, or any combination of two or more thereof.

According to some embodiments, the feedstock includes a lignocellulosic derived material.

In various embodiments of the methods of the invention, the feedstock can include a lignocellulosic derived material, such as material derived at least in part from biomass and/or lignocellulosic sources.

The growing and/or consuming can be carried under suitable conditions, including various temperatures. In some embodiments the growing and/or consuming can be carried out at a temperature of at least about 18° C., at least about 20° C., at least about 25° C., at least about 30° C., at least about 35° C., at least about 40° C., at least about 45° C., or at least about 50° C. In some embodiments the growing and/or consuming can be carried out at a temperature of less than about 100° C. In some embodiments the growing and/or consuming can be carried out at a temperature of about 18° C. to about 50° C., about 20° C. to about 30° C., and/or the like. Operating at high temperatures can increase yield, increase productivity, reduce growth of foreign organisms, and/or the like. Consuming and/or producing can occur at a same temperature and/or a different temperature. Embodiments with temperature transients (change in temperature with respect to time) during the consuming and/or producing are within the scope of the invention.

According to some embodiments, the method and/or process can include temperature control, such as by addition of heat, cooling, and/or the like. Heat can be supplied by steam, saturated stream, super heated stream, hot water, glycol, heat transfer oil, heat transfer fluid, other process streams, and/or the like. Cooling can be supplied by cooling water, refrigerant, brine, glycol, heat transfer fluid, coolant, other process streams, and/or the like. Temperature control can use any suitable technique and/or configuration, such as indirect heat exchange, direct heat exchange, convection, conduction, radiation, and/or the like.

According to some embodiments of the method and/or processes, the growing comprises consuming of a feedstock by the culture at a temperature of at least about 20° C.

In various embodiments of the method and/or processes, the growing and/or consuming is carried out under nitrogen limiting conditions. Nitrogen limitation refers to lacking in nitrogen, such as used in cellular reproduction. In the alternative, at least a portion of consuming and/or growing occur with nitrogen addition, such as from ammonia.

Under nitrogen limiting conditions (favoring oil production), certain microorganisms, e.g., yeast, can reduce and/or stop utilizing a fructose portion of sucrose (glucose+fructose). Glycerol addition to a fermentation medium allows the microorganism to start utilizing fructose from the carbon source, such as sucrose. Similarly, increasing fermentation temperature, decreasing pH, and decreasing nitrogen levels can favorably lower production cost while increasing oil production. In other embodiments, at least 10 percent of total nitrogen, at least about 20 percent total nitrogen, at least about 40 percent of total nitrogen, and/or at least about 50 percent of total nitrogen can be supplied to the fermentation as part of the carbon source feedstock during the production of an oil.

According to some embodiments of the method and/or processes, the feedstock used in the growing or consuming includes at least one organic acid.

According to some embodiments of the method and/or processes of the invention, feedstock comprises sugar and glycerol.

According to some embodiments, the growing and/or consuming is carried out at a pH of about 8 or below.

Other materials and/or substances can be used in the methods and/or processes of the invention to aid and/or assist the organism, such as nutrients, vitamins, minerals, metals, water, and/or the like. The use of other additives is also within the scope of this invention, such as antifoam, flocculants, emulsifiers, demulsifiers, viscosity modifiers, surfactants, salts, other fluid modifying materials, and/or the like.

In various embodiments, the methods and/or processes further include extracting fatty acids from the organism, typically as fatty acid triglycerides. The extracting may be solvent extraction using a chemical solvent, physical solvent, non-polar solvent, polar solvent, supercritical solvent, or combinations thereof. Representative solvents for include ethyl acetate, an alcohol such as ethanol, or a hydrocarbon such as petroleum ether, pentane, hexane, or the like, or combinations thereof.

The methods and/or processes may include other suitable actions, such as removing the fatty acids by cell lysing, applying pressure, distillation, centrifugation, other mechanical processing, other thermal processing, other chemical processing, and/or the like. In the alternative, the producing organism can secrete or excrete and/or discharge the lipid containing fatty acids from the organism without additional processing.

After growing in the presence of a carbon source and extracting a product comprising fatty acid triglycerides, the fatty acid triglycerides, in some embodiments of the methods of the invention, are converted to a biofuel, e.g., a fatty acid alkyl ester composition, particularly a fatty acid C₁-C₄ alkyl ester composition, and more particularly a fatty acid methyl ester composition, by hydrolysis, esterification, transesterification, hydrogenation, or combinations thereof.

According to some embodiments, the invention is directed to a biofuel made from the any of the oils, fatty acid triglyceride compositions, or fatty acid ester compositions, disclosed within this specification.

The oil or triglyceride composition can be further processed into the biofuel with any suitable method, such as esterification, transesterification, hydrogenation, cracking, and/or the like. In the alternative, the biological oil can be suitable for direct use as a biofuel. Esterification refers to making and/or forming an ester, such as by reacting an acid with an alcohol to form an ester, Transesterification refers to converting one ester into one or more different esters, such as, for example, by reaction of an alcohol with a triglyceride to form fatty acid esters and glycerol. Hydrogenation and/or hydrotreating refer to reactions to add hydrogen to molecules, such as to saturate and/or reduce materials.

Transesterification can include use of any suitable alcohol, such as methanol, ethanol, propanol, butanol, and/or the like.

The growing and/or consuming can occur at any suitable pH, such as a pH of below about 3, a pH of below about 5, a pH of below about 6, a pH of about 7.0 or below, a pH of about 7, a pH of at least about 8, a pH of at least about 9, a pH of at least about 10, a pH of about 5 to about 9, a pH of about 6 to about 8, a pH of about 7 to about 8, and/or the like. Operating at different pH levels can inhibit growth of foreign organisms. Inhibiting growth of foreign organisms can allow for operation of the method to occur without a need for a separate sterilization process, such as at beginning of each batch. Embodiments with changes in pH during operation are within the scope of the invention.

Sterilization can consume energy, time, and/or other resources. Therefore, in some embodiments, a sterilization process is not utilized, for example when the pH level inhibits growth of foreign organisms, as discussed above. In the alternative, the method can further include a sterilization process, such as steaming to at least a threshold temperature for a suitable duration.

In certain embodiments, the invention is directed to methods for producing a feedstock suitable for use in the production of a biofuel. These methods comprise growing a culture of an oleaginous microorganism in a presence of a carbon source, wherein material produced by the culture comprises substantially no sterol glycosides.

In some embodiments, the methods for producing a feedstock further comprise isolating an oil from the culture. In some embodiments, the isolating comprises lysing the microorganism.

In some embodiments, the methods for producing a feedstock employ the organisms or cultures of one or more microorganisms described above.

As noted above, the invention provides methods for producing a feedstock suitable for use in the production of a biofuel. These methods comprise growing a culture of at least one oleaginous microorganism in a presence of a carbon source, wherein material produced by the culture comprises substantially no sterol glycosides.

As also noted above, the invention provides methods for producing a fatty acid derivative composition. The fatty acid derivative may be a fatty acid triglyceride or fatty acid alkyl ester. These methods comprise growing a culture of at least one oleaginous microorganism in the presence of a carbon source or feedstock, wherein material produced by the culture comprises substantially no sterol glycosides. In certain embodiments, these methods further comprise lysing the microorganism to produce a lysate. These methods may further comprise isolating an oil from the lysate. These methods employ the organisms or cultures of one or more microorganisms described herein.

In some embodiments, the methods for producing a fatty acid derivative composition further comprise hydrolyzing the oil to produce a hydrolysis product comprising one or more acids, one or more acid salts, or combinations thereof.

In some embodiments, the methods for producing a fatty acid derivative composition further comprise esterifying the hydrolysis product with an alcohol comprising about one to about four carbon atoms.

In some embodiments, the methods of the invention do not employ processing to reduce sterol glycosides levels.

According to some embodiments, the invention is directed to a system or unit for producing an oil, particularly a biological oil, which oil may comprise fatty acid triglycerides. The system or unit may include a feedstock stream, a vessel connected to the feedstock stream, an organism disposed within the vessel, and/or a lipid containing stream, e.g., a fatty acid triglyceride containing stream, connected to the vessel. Alternatively, the system or unit comprises a fatty acid containing stream connected to the vessel.

According to some embodiments, the vessel operates on a batch basis, a discrete basis, a semi-batch basis, a semi-continuous basis, a continuous basis, and/or the like. Combinations of series and/or parallel vessels are within the scope of the invention.

In certain embodiments, the unit can be constructed such that the feedstock is fed into the unit for use in the methods of the invention using one or more feeds. In some embodiments, a feedstock can be present in media charged to a vessel prior to inoculation with the culture of one or more microorganisms. In some embodiments, a feedstock can be added through one or more feed streams in addition to the media charged to the vessel.

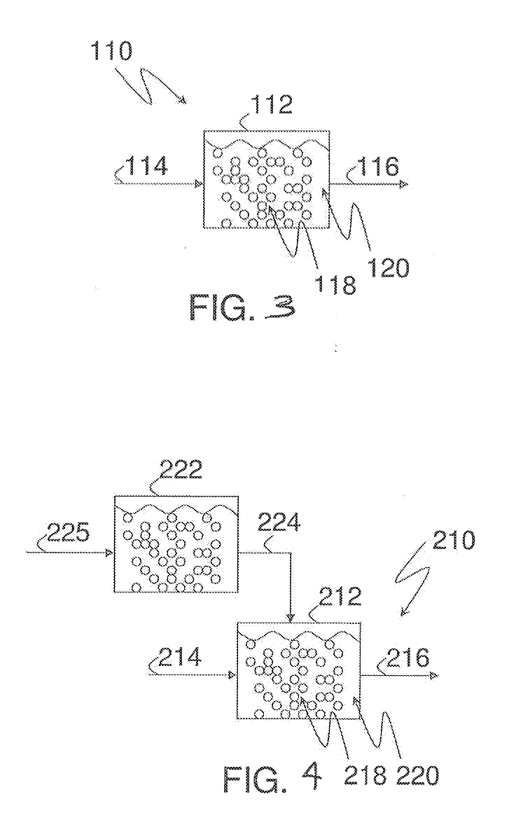

Examples of systems and units useful for carrying out the methods of the invention are shown in FIGS. 3-6.

FIG. 3 schematically shows a system or unit 110 suitable for use in the methods described herein. Unit 110 includes a vessel 112 with a feedstock stream 114 connected to the vessel 112 and a lipid (fatty acids) stream 116 connected to the vessel 112. The feedstock stream 114 provides feedstock to the vessel 112, wherein the feedstock can be any of the materials and/or substances included in the definition of feedstock below. Lipids present in the vessel 112 exit the vessel 112 through the lipid stream 116, wherein the lipids can be any of the substances included in the definition of lipids below. The vessel 112 includes or contains an organism 18 disposed within the vessel 112, wherein the organism 118 can be any of the substances included in the definition of organism below. The vessel 112 includes or contains a medium 120, such as a fermentation broth. The organism 118 can be in the medium 120.

FIG. 4 schematically shows a two stage unit 210 suitable for use in the methods described herein. The two stage unit 210 includes a vessel 212 with a feedstock stream 214 connected to the vessel 212 and a lipid (fatty acids) stream 216 connected to the vessel 212. The feedstock stream 214 provides feedstock to the vessel 212, wherein the feedstock can be any of the materials and/or substances included in the definition of feedstock below. Lipids present in the vessel 212 exit the vessel 212 through the lipid stream 216, wherein the lipids can be any of the substances included in the definition of lipids below. The vessel 212 includes or contains an organism 218 disposed within the vessel 212, wherein the organism 218 can be any of the substances included in the definition of organism below. The vessel 212 includes or contains a medium 220, such as a fermentation broth. The organism 218 can be in the medium 220. The two stage unit 210 includes a growth vessel 222 with a growth feedstock stream 225 and an organism stream 224 that connects the growth vessel 222 to the vessel 212. The growth feedstock stream 225 provides growth feedstock to the growth vessel 222, wherein the growth feedstock can be the same feedstock present in the feedstock stream 214. The organism stream 224 provides the organism 218 from the growth vessel 222 to the vessel 212.

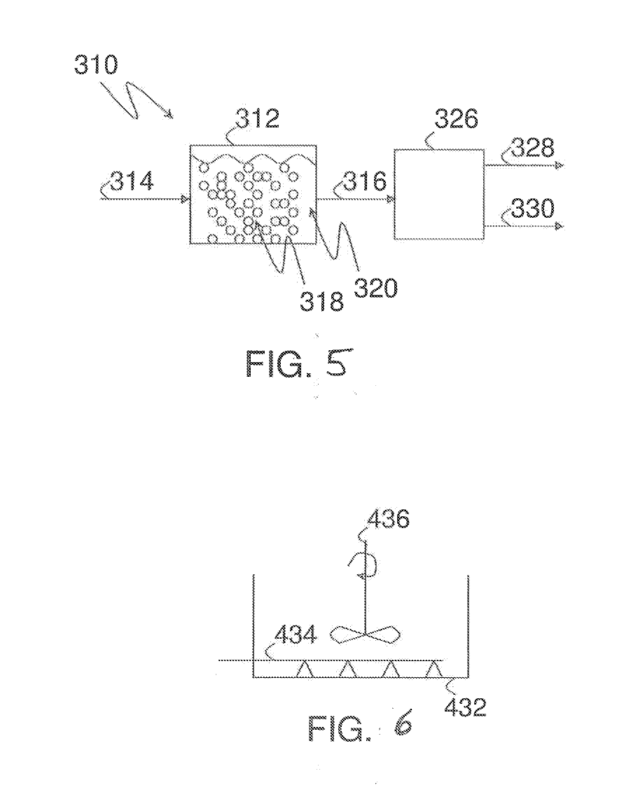

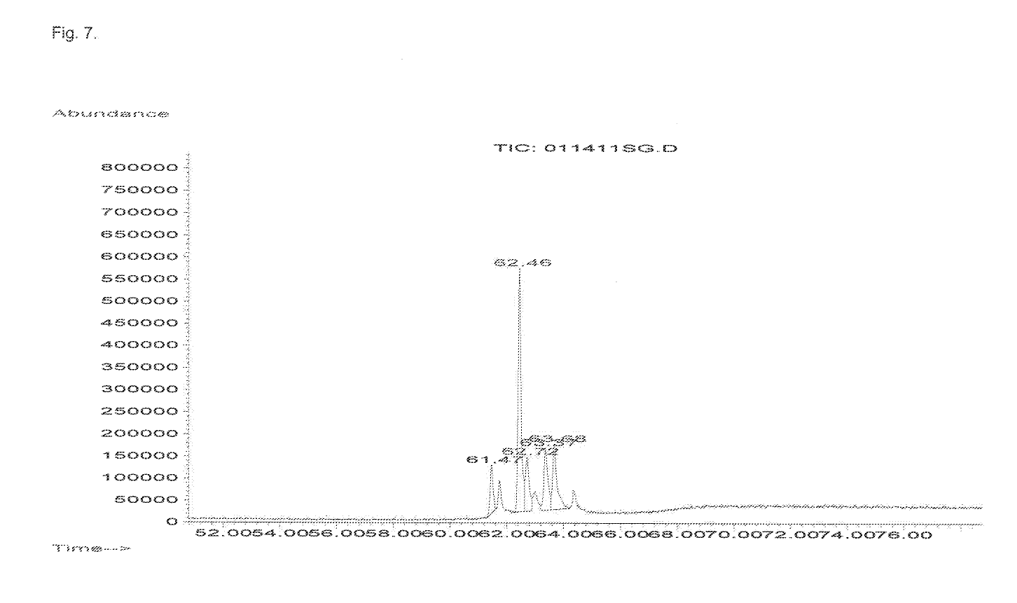

FIG. 5 schematically shows a unit 310 with extraction suitable for use in the methods described herein. The unit 310 includes a vessel 312 with a feedstock stream 314 connected to the vessel 312 and a lipid (fatty acids) stream 316 connected to the vessel 312. The feedstock stream 314 provides feedstock to the vessel 312, wherein the feedstock can be any of the materials and/or substances included in the definition of feedstock below. Lipids present in the vessel 312 exit the vessel 312 through the lipid stream 316, wherein the lipids can be any of the substances included in the definition of lipids below. The vessel 312 includes or contains an organism 318 disposed within the vessel 312, wherein the organism 218 can be any of the substances included in the definition of organism below. The vessel 312 includes or contains a medium 320, such as a fermentation broth. The organism 318 can be in the medium 320. The unit 310 includes an extraction apparatus 326. The lipid stream 316 is fed to the extraction apparatus 326 from the vessel 312. The extraction apparatus 326 removes the lipids present in lipid stream 316 from the remainder of the contents of the lipid stream 316 so that a lipid product stream 328 exits the extraction apparatus 326. A delipidated biomass stream 330 also exits the extraction apparatus 326.

FIG. 6 schematically shows a fermentor 432 suitable for use in the methods described herein. The fermentor 432 includes a sparger 434, such as for introduction of air and/or other gases into a process. The fermentor 432 includes an agitator 436, such as for stirring contents of the fermentor 432. In some embodiments, the vessel 112, the vessel 212, or the vessel 312 can be a fermentor similar to the fermentor 432.

### DEFINITIONS

Adapted refers to make fit for a specific use, purpose, and/or the like.

As used herein the terms “has”, “having”, “comprising”, “with”, “containing”, and “including” are open and inclusive expressions. Alternately, the term “consisting” is a closed and exclusive expression. Should any ambiguity exist in construing any term in the claims or the specification, the intent of the drafter is toward open and inclusive expressions.

As used herein the term “and/or the like” provides support for any and all individual and combinations of items and/or members in a list, as well as support for equivalents of individual and combinations of items and/or members.

Regarding an order, number, sequence, omission, and/or limit of repetition For steps in a method or process, the drafter intends no implied order, number, sequence, omission, and/or limit of repetition for the steps to the scope of the invention, unless explicitly provided.

Regarding ranges, ranges are to be construed as including all points between upper values and lower values, such as to provide support for all possible ranges contained between the upper values and the lower values including ranges with no upper bound and/or lower bound.

The steps or parts of a method or process may be carried out in any order unless otherwise specified.

The basis for operations, percentages, and procedures can be on any suitable basis, such as a mass basis, a volume basis, a mole basis, and/or the like. If a basis is not specified, a mass basis or other appropriate basis should be used.

Biomass refers to plant and/or animal materials and/or substances derived at least in part from living organisms and/or recently living organisms, such as plants and/or lignocellulosic sources, Biomass can include other materials and/or substances to aid and/or assist the organism, such as nutrients, vitamins, minerals, metals, water, and/or the like.

Density refers to a mass per unit volume of a material and/or a substance. Cell density refers to a mass of cells per unit volume, such as the weight of living cells per unit volume. It is commonly expressed as grams of dry cells per liter. The cell density can be measured at any suitable point in the method, such as upon commencing fermentation, during fermentation, upon completion of fermentation, over the entire batch, and/or the like.

Content refers to an amount of specified material contained. Dry mass basis refers to being at least substantially free from water. The fatty acid content can be measured at any suitable point in the method, such as upon commencing fermentation, during fermentation, upon completion of fermentation, over the entire batch, and/or the like.

Productivity refers to a measurement of a quality and/or characteristic of producing and/or making, such as a rate per unit of volume. For example, fatty acid productivity can be measured at any suitable point in the method, such as upon commencing fermentation, during fermentation, upon completion of fermentation, over the entire batch, and/or the like. Fatty acid productivity can be measured on a fixed time, such as noon to noon each day. In the alternative, fatty acid productivity can be measured on a suitable rolling basis, such as for any 24 period.

Yield refers to an amount and/or quantity produced and/or returned. The fatty acid yield can be measured at any suitable point in the method, such as upon commencing fermentation, during fermentation, upon completion of fermentation, over the entire batch, and/or the like.

Growing refers to increasing in size, such as by assimilation of material into the living organism and/or the like. Growing also refers to increasing the number of something, e.g., microorganisms. Thus, for example, growing a culture of an oleaginous microorganism encompasses increasing the number of microorganisms in the culture.

Biological oils refer to hydrocarbon containing materials (including heteroatoms) and/or substances derived at least in part from living organisms, such as animals, plants, fungi, yeasts, algae, microalgae, bacteria, and/or the like. According to some embodiments, biological oils can be suitable for use as and/or conversion into renewable materials and/or biofuels. In some embodiments, biological oils refer to triglycerides and related compounds.

Biofuel refers to components and/or streams suitable for use as a fuel and/or a combustion source derived at least in part from renewable sources. The biofuel can be sustainably produced and/or have reduced and/or no net carbon emissions to the atmosphere, such as when compared to fossil fuels. According to some embodiments, renewable sources can exclude materials mined or drilled, such as from the underground. In some embodiments, renewable resources can include single cell organisms, multicell organisms, plants, fungi, bacteria, algae, cultivated crops, non-cultivated crops, timber, and/or the like. Biofuels can be suitable for use as transportation fuels, such as for use in land vehicles, marine vehicles, aviation vehicles, and/or the like. Biofuels can be suitable for use in power generation, such as raising steam, exchanging energy with a suitable heat transfer media, generating syngas, generating hydrogen, making electricity, and or the like.

Biodiesel refers to components or streams suitable for direct use and/or blending into a diesel pool and/or a cetane supply derived from renewable sources. Suitable biodiesel molecules can include fatty acid esters, monoglycerides, diglycerides, triglycerides, lipids, fatty alcohols, alkanes, naphthas, distillate range materials, paraffinic materials, aromatic materials, aliphatic compounds (straight, branched, and/or cyclic), and/or the like. Biodiesel can be used in compression ignition engines, such as automotive diesel internal combustion engines, truck heavy duty diesel engines, and/or the like. In the alternative, the biodiesel can also be used in gas turbines, heaters, boilers, and/or the like. According to some embodiments, the biodiesel and/or biodiesel blends meet or comply with industrially accepted fuel standards, such as B20, B40, B60, B80, B99.9, B100, and/or the like.

Biodistillate refers to components or streams suitable for direct use and/or blending into aviation fuels (jet), lubricant base stocks, kerosene fuels, fuel oils, and/or the like. Biodistillate can be derived from renewable sources, and have any suitable boiling point range, such as a boiling point range of about 100° C. to about 700° C., about 150° C. to about 350° C., and/or the like.

Consuming refers to using up, utilizing, eating, devouring, transforming, and/or the like. According to some embodiments, consuming can include processes during and/or a part of cellular metabolism (catabolism and/or anabolism), cellular respiration (aerobic and/or anaerobic), cellular reproduction, cellular growth, fermentation, cell culturing, and/or the like.

Fatty acid as used herein refers to carboxylic acids having straight or branched hydrocarbon groups having from about 8 to about 30 carbon atoms. The hydrocarbon groups including from 1 to about 4 sites of unsaturation, generally double or pi bonds. Examples of such fatty acids are lauric acid, steric acid, palmitic acid, oleic acid, linoleic acid, linolenic acid, arachidonic acid, elaidic acid, linoelaidicic acid, eicosenoic acid, phytanic acid, behenic acid, and adrenic acid.

Feedstock refers to materials and/or substances used to supply, feed, provide for, and/or the like, such as to an organism, a machine, a process, a production plant, and/or the like. Feedstocks can include raw materials used for conversion, synthesis, and/or the like. According to some embodiments, the feedstock can include any material, compound, substance, and/or the like suitable for consumption, for example, by an organism, such as sugars, hexoses, pentoses, monosaccharides, disaccharides, trisaccharides, polyols (sugar alcohols), organic acids, starches, carbohydrates, and/or the like. According to some embodiments, the feedstock can include sucrose, glucose, fructose, xylose, glycerol, mannose, arabinose, lactose, galactose, maltose, other five carbon sugars, other six carbon sugars, other twelve carbon sugars, plant extracts containing sugars, other crude sugars, and/or the like. Feedstock can refer to one or more of the organic compounds listed above when present in the feedstock.

Fermentation refers both to cell culturing and to metabolism of carbohydrates where a final electron donor is not oxygen, such as anaerobically. Fermentation can include an enzyme controlled anaerobic breakdown of an energy rich compound, such as a carbohydrate to carbon dioxide and an alcohol, an organic acid, a lipid, and/or the like. In the alternative, fermentation refers to biologically controlled transformation of an inorganic or organic compound. Fermentation processes can use any suitable organisms, such as bacteria, fungi (including yeast), algae, and/or the like. Suitable fermentation processes can include naturally occurring organisms and/or genetically modified organisms.

Glyceride refers to glycerol esters of acids and includes monoglycerides, diglycerides, triglycerides, and/or the like, where the acids may be the same or different.

Lipid refers to oils, fats, waxes, greases, cholesterol, glycerides, steroids, phosphatides, cerebrosides, fatty acids, fatty acid related compounds, derived compounds, other oily substances, and/or the like. Lipids can be made in living cells and can have a relatively high carbon content and a relatively high hydrogen content with a relatively lower oxygen content. Lipids typically include a relatively high energy content, such as on a mass basis.

Lignocellulosic refers to containing at least some cellulose, hemicellulose, lignin, and/or the like. Lignocellulosic can refer to plant and/or plant derived material. Lignocellulosic material can include any suitable material, such as sugar cane, sugar cane bagasse, energy cane, energy cane bagasse, rice, rice straw, corn, corn stover, wheat, wheat straw, maize, maize stover, sorghum, sorghum stover, sweet sorghum, sweet sorghum stover, cotton, cotton remnant, cassaya, sugar beet, sugar beet pulp, soybean, rapeseed, jatropha, switchgrass, miscanthus, other grasses, timber, softwood, hardwood, wood bark, wood waste, sawdust, paper, paper waste, agricultural waste, manure, dung, sewage, municipal solid waste, any other suitable biomass material, and/or the like. Lignocellulosic material can be pretreated and/or treated by any suitable process and/or method, such as acid hydrolysis, neutral hydrolysis, basic hydrolysis, thermal hydrolysis, catalytic hydrolysis, enzymatic hydrolysis, ammonia fiber expansion, steam explosion, and/or the like.

Naturally occurring refers to organisms, cultures, single cells, biota, and/or the like at least generally without intervening actions by exterior forces, such as humankind, machine, and/or the like. Naturally occurring organisms can include those found in local environments (flora and/or fauna) and/or the like. Naturally occurring organisms can be collected, isolated, cultured, purified, and/or the like.

Organic refers to carbon containing compounds, such as carbohydrates, sugars, ketones, aldehydes, alcohols, lignin, cellulose, hemicellulose, pectin, other carbon containing substances, and/or the like.

The organism can include any suitable simple (mono) cell being, complex (multi) cell being, and/or the like. Organisms can include algae, fungi (including yeast), bacteria, and/or the like. The organism can include microorganisms, such as bacteria or protozoa. The organism can include one or more naturally occurring organisms, one or more genetically modified organisms, combinations of naturally occurring organisms and genetically modified organisms, and/or the like. Embodiments with combinations of multiple different organisms are within the scope of the invention. Any suitable combination or organism can be used, such as one or more organisms, at least about two organisms, at least about five organisms, about two organisms to about twenty organisms, and/or the like.

Oil refers to hydrocarbon substances and/or materials that are at least somewhat hydrophobic and/or water repelling. Oil can include mineral oil, organic oil, synthetic oil, essential oil, and/or the like. Mineral oil refers to petroleum and/or related substances derived at least in part from the Earth and/or underground, such as fossil fuels. Organic oil refers to materials and/or substances derived at least in part from plants, animals, other organisms, and/or the like. Synthetic oil refers to materials and/or substances derived at least in part from chemical reactions and/or processes, such as can be used in lubricating oil. Oil can be at least generally soluble in nonpolar solvents and other hydrocarbons, but at least generally insoluble in water and/or aqueous solutions. Oil can be at least about 50 percent soluble in nonpolar solvents, at least about 75 percent soluble in nonpolar solvents, at least about 90 percent soluble in nonpolar solvents, completely soluble in nonpolar solvents, about 50 percent soluble in nonpolar solvents to about 100 percent soluble in nonpolar solvents and/or the like, all on a mass basis.

Oleaginous microorganism refers to microorganisms that are oil-bearing or oil-containing, or that are capable of producing oils, lipids, fats, and/or other oil-like substances. Oleaginous microorganisms may include organisms that produce at least about 20 percent by weight of oils, at least about 30 percent by weight of oils, at least about 40 percent by weight oils, at least about 50 percent by weight oils, at least about 60 percent by weight oils, at least about 70 percent by weight oils, at least about 80 percent by weight oils, based on the dry weight of the microorganism, and/or the like. These amounts refer to amounts of oil etc. accumulated in the microorganism as well as amounts of oil etc. both accumulated and secreted by the microorganism.

Organism refers to an at least relatively complex structure of interdependent and subordinate elements whose relations and/or properties can be largely determined by their function in the whole. The organism can include an individual designed to carry on the activities of life with organs separate in function but mutually dependent. Organisms can include a living being, such as capable of growth, reproduction, and/or the like.

The organism can include any suitable simple (mono) cell being, complex (multi) cell being, and/or the like. Organisms can include algae, fungi (including yeast), bacteria, and/or the like. The organism can include one or more naturally occurring organisms, one or more genetically modified organisms, combinations of naturally occurring organisms and genetically modified organisms, and/or the like. Embodiments with combinations of multiple different organisms are within the scope of the invention. Any suitable combination or organism can be used, such as one or more organisms, at least about two organisms, at least about five organisms, about two organisms to about twenty organisms, and/or the like.

Producing and production refer to making, forming, creating, shaping, bringing about, bringing into existence, manufacturing, growing, synthesizing, and/or the like. According to some embodiments, producing includes fermentation, cell culturing, and/or the like. Producing can include new or additional organisms as well as maturation of existing organisms.

Renewable materials refer to substances and/or items that have been at least partially derived from a source and/or process capable of being replaced by natural ecological cycles and/or resources. Renewable materials can include chemicals, chemical intermediates, solvents, monomers, oligomers, polymers, biofuels, biofuel intermediates, biogasoline, biogasoline blendstocks, biodiesel, green diesel, renewable diesel, biodiesel blend stocks, biodistillates, and/or the like. In some embodiments, the renewable material can be derived from a living organism, such as plants, algae, bacteria, fungi, and/or the like.

Sterol glycoside refers to molecules having a sugar moiety covalently bound to a steroid group by a glycosidic linkage.

Sterol glucoside refers to molecules having a glucose molecule covalently bound to a steroid group by a glycosidic linkage. Steroids typically comprise a gonane ring system; the gonane ring system is a fused tetracycle and can be represented by the following structure:

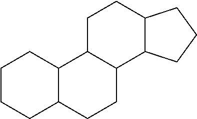 [SMILES: C1CCC2C(C1)CCC1C3CCCC3CCC21]

Thus, many sterol glucosides can be represented by the following structure (stereochemistry, unsaturation, and optional alkyl and hydroxy side chains on the gonane not shown for convenience):

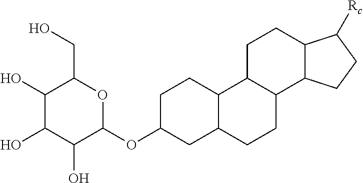

where R_{c} represents an alkyl or alkylene group.
Examples of the steroid groups in sterol glycosides and glucosides are sitosterol, cholesterol, ergosterol, dihydroergosterol, poriferasterol, campesterol, and stigmasterol.

Stream refers to a flow and/or a supply of a substance and/or a material, such as a steady succession. Flow of streams can be continuous, discrete, intermittent, batch, semi-batch, semi-continuous, and/or the like.

System refers to a device or apparatus suitable for carrying out a process. System also refers to a process for producing a desired result.

Unit refers to a single quantity regarded as a whole, a piece and/or complex of apparatus serving to perform one or more particular functions and/or outcomes, and/or the like.

Vessel refers to a container and/or holder of a substance, such as a liquid, a gas, a fermentation broth, and/or the like. Vessels can include any suitable size and/or shape, such as at least about 1 liter, at least about 1,000 liters, at least about 100,000 liters, at least about 1,000,000 liters, at least about 1,000,000,000 liters, less than about 1,000,000 liters, about 1 liter to about 1,000,000,000 liters, and/or the like. Vessels can include tanks, reactors, columns, vats, barrels, basins, and/or the like. Vessels can include any suitable auxiliary equipment, such as pumps, agitators, aeration equipment, heat exchangers, coils, jackets, pressurization systems (positive pressure and/or vacuum), control systems, and/or the like.

### EXAMPLES

### Example 1

A strain of the yeast Sporidiobolus pararoseus (ATCC 11616), as confirmed as a one hundred percent DNA match to several strains of Sporidiobolus pararoseus, was cultivated in a 14 liter New Brunswick Scientific BioFlo 3000 fermentor with a carbon (sucrose syrup) and nitrogen (ammonium hydroxide) fed-batch process. The fermentation was inoculated with 0.75 liters of inoculum culture. For inoculum propagation a 3 liter Broadley James BioNet fermentor was utilized. The inoculum medium included 2.3 liters of medium prepared in four separate groups. Group A included 20.7 grams MSG*1H₂O, 1.44 grams NaCl, 0.667 grams CaCl₂*2H₂O, 2.3 grams KCl, 11.5 grams MgSO₄*7H₂O, 0.85 grams (NH₄)₂SO₄, 13.8 grams yeast extract (T154), 1.196 grams KH₂PO₄, and 0.23 milliliters Dow 1520US (antifoam). Group A was autoclaved at 121 degrees in the inoculum fermentor at a volume of approximately 2.1 liters. Group B included 23.7 milligrams FeSO₄*7H₂O, 42.412 milligrams citric acid, 7.13 milligrams MnCl₂*4H₂O, 7.13 milligrams ZnSO₄*7H₂O, 0.092 milligrams CoCl₂*6H₂O, 0.092 milligrams Na₂MoO₄*2H₂O, 4.761 milligrams CuSO₄*5H₂O, and 4.761 milligrams NiSO₄*6H₂O in a volume of 4.5 milliliters of distilled water. The group B stock solution was autoclaved at 121° C. Group C included 22.425 milligrams thiamine-HCl, 0.368 milligrams vitamin B12, and 7.67 milligrams pantothenic acid hemi-calcium salt dissolved in approximately 2 milliliters distilled water and filter sterilized. Group D included 200 milliliters of distilled water containing 115 grams sucrose powder. After the fermentor was cooled to 30° C., groups B, C, and D were added to the fermentor. Using sodium hydroxide and sulfuric acid, the fermentor was pH adjusted to 6.9 and the dissolved oxygen was spanned to 100 percent prior to inoculation. The inoculum fermentor was inoculated with 29.5 milliliters of a standard shake flask culture and cultivated at 30° C., pH 6.9, 847 revolutions per minute agitation, and 1.2 liters per minute of air for a period of 14.5 hours, at which point 0.75 liters was harvested from the fermentor and transferred to the 14 liter fermentor. The 14 liter fermentor included 10 liters of fermentation media. The fermentation media was prepared in a similar fashion to the inoculum fermentor. The fermentation media included 4 batched media groups. Group A included 6.25 grams NaCl, 4.2 grams (NH₄)₂SO₄, 10 grams yeast extract (T154), 12.66 grams Na₂HPO₄, and 1.0 milliliters Dow 1520US (antifoam). Group A was autoclaved at 121° C. in the fermentor at a volume of approximately 6.25 liters. Group B included 103 milligrams FeSO₄*7H₂O, 370 milligrams citric acid, 31 milligrams MnCl₂*4H₂O, 31 milligrams ZnSO₄*7H₂O, 0.4 milligrams CoCl₂*6H₂O, 0.4 milligrams Na₂MoO₄.2H₂O, 20.7 milligrams CuSO₄*5H₂O, and 20.7 milligrams NiSO₄*6H₂O in a volume of approximately 45 milliliters of distilled water. The group B stock solution was autoclaved at 121° C. Group C included 97.5 milligrams thiamine-HCl, 1.6 milligrams vitamin B12, 33.3 milligrams pantothenic acid hemi-calcium salt, and 35.8 micrograms biotin dissolved in approximately 10 milliliters and filter sterilized. Group D included approximately 700 milliliters of sugar syrup obtained from Raceland Raw Sugar Corporation in Louisiana, U.S.A. After the fermentor was cooled to 27° C., groups B, C, and D were added to the fermentor. Using sodium hydroxide and sulfuric acid, the fermentor was pH adjusted to 7 and the dissolved oxygen was spanned to 100 percent prior to inoculation. The fermentor volume prior to inoculation was approximately 6.15 liters.

The fermentor was inoculated with 0.75 liters of broth from the inoculum fermentation described above. The fermentation was pH controlled utilizing a 0.26 liter solution of 6N ammonium hydroxide at a pH of 7. The dissolved oxygen was controlled to maintain a target set point of 20 percent throughout the fermentation using agitation from 357 revolutions per minute to 1200 revolutions per minute, airflow from 3 liters per minute to 8 liters per minute, and oxygen from 0 liters per minute to 5 liters per minute. Throughout the fermentation, 5.65 liters of (Raceland) sugar syrup was fed to maintain a total sugar (glucose+fructose+sucrose) concentration less than 80 grams per liter. After 92 hours, the fermentor included 1251 grams of biomass that included 670.7 grams of fatty acids. The final cell density was 125.1 grams per liter dry weight. The fatty acid content was 53.63 percent of cellular dry weight, the average fatty acid productivity was 17.56 grams per liter per day and the resulting fatty acid yield (grams of fatty acids produced per grams of carbon feedstock) of the culture was 0.1625. The highest peak fatty acid productivity of the culture, measured over an 8 hour period was 56.8 grams per liter per day. The highest peak fatty acid productivity of the culture measured over a 24 hour period was 30.5 grams per liter per day. The fatty acid yield (grams of fatty acid produced per grams of carbon feedstock) at 27° C., pH 7 and 0.5× nitrogen was 0.1625.

Analysis for sterol glycoside levels is carried out essentially according to the following procedure.

Samples are prepared by dissolving approximately 1 mL of lipid material with an equal volume of N,O-bis-(trimethylsilyl)-trifluoroacetamide (BSTFA) silylation reagent. Silylation solutions are warmed to approximately 50° C. and allowed to react for 30 minutes. The silylated material is diluted 1:10 with dichloromethane and analyzed using the following GC/MS conditions.

| | |
|---|---|
| Column | 15 m DB5-MS × 0.32 mm, |
| | 0.10 um film thickness |
| Injection Volume, Type | 1 uL, splitless |
| Flow Rate, Head Pressure | 4 mL/min, 9.5 psig |
| Oven Program | 50° C. to 325° C. at 4° C./min |
| Carrier Gas | Helium |
| Pressure Mode | Constant Pressure |
| Mass Range, Ionization | 40-900, El 70 eV |

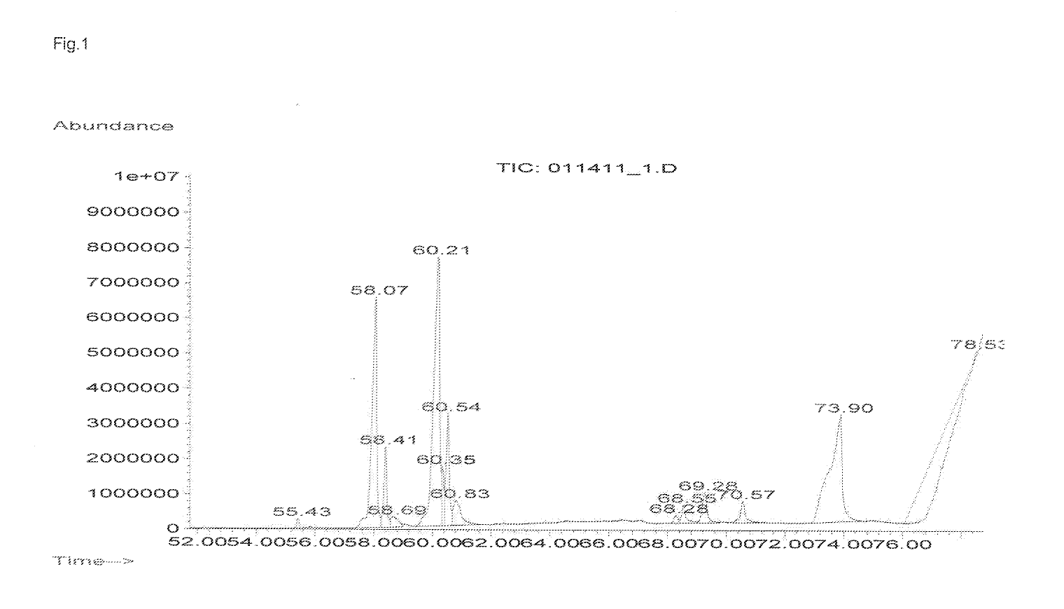

Results of GC-MS analysis for the product of this example are shown in FIG. 1.

### Example 2

Yeast Rhodotorula ingenosa (ATCC 11617), as confirmed as a ninety-nine percent DNA match to Rhodotorula ingenosa, was cultivated in a 14 liter New Brunswick Scientific BioFlo 3000 fermentor with a carbon (sucrose syrup) and nitrogen (ammonium hydroxide) fed-batch process. The fermentation was inoculated with 0.75 liters of inoculum culture. For inoculum propagation a 3 liter Broadley James BioNet fermentor was utilized. The inoculum medium included 2.2 liters of medium prepared in four separate groups. Group A included 207 grams MSG*1H₂O, 1.44 grams NaCl, 0.667 grams CaCl₂*2H₂O, 2.3 grams KCl, 11.5 grams MgSO₄*7H₂O, 0.85 grams (NH₄)₂SO₄, 13.8 grams yeast extract (T154), 1.196 grams KH₂PO₄, and 0.23 milliliters Dow 1520US (antifoam). Group A was autoclaved at 121 degrees in the inoculum fermentor at a volume of approximately 2.15 liters. Group B included 23.7 milligrams FeSO₄*7H₂O, 42.412 milligrams citric acid, 7.13 milligrams MnCl₂*4H2O, 7.13 milligrams ZnSO₄*7H₂O, 0.092 milligrams CoCl₂*6H₂O, 0.092 milligrams Na₂MoO₄*2H₂O, 4.761 milligrams CuSO₄*5H₂O, and 4.761 milligrams NiSO₄*6H₂O in a volume of 52 milliliters of distilled water. The group B stock solution was autoclaved at 121° C. Group C included 22.425 milligrams thiamine-HCl, 0.368 milligrams vitamin B12, and 7.67 milligrams pantothenic acid hemi-calcium salt dissolved in approximately 2 milliliters distilled water and filter sterilized. Group D included 200 milliliters of distilled water containing 115 grams sucrose powder. After the fermentor was cooled to 27° C., groups B, C, and D were added to the fermentor. Using sodium hydroxide and sulfuric acid, the fermentor was pH adjusted to 5.09 and the dissolved oxygen was spanned to 100 percent prior to inoculation.

The inoculum fermentor was inoculated with 23 milliliters of a standard shake flask culture and cultivated at 27° C., pH 5.09, 644 revolutions per minute agitation, and 1.2 liters per minute of air for a period of 17.17 hours, at which point 0.75 liters was harvested from the fermentor and transferred to the 14 liter fermentor. The 14 liter fermentor included 10 liters of fermentation media. The fermentation media was prepared in a similar fashion to the inoculum fermentor. The fermentation media included 4 batched media groups. Group A included 6.25 grams NaCl, 4.2 grams (NH₄)₂SO₄, 10 grams yeast extract (T154), 12.66 grams Na₂HPO₄, and 1.0 milliliters Dow 1520US (antifoam). Group A was autoclaved at 121° C. in the fermentor at a volume of approximately 6.25 liters. Group B included 103 milligrams FeSO₄*7H₂O, 370 milligrams citric acid, 31 milligrams MnCl₂*4H₂O, 31 milligrams ZnSO₄*7H₂O, 0.4 milligrams CoCl₂*6H₂O, 0.4 milligrams Na₂MoO₄*2H₂O, 20.7 milligrams CuSO₄*5H₂O, and 20.7 milligrams NiSO₄*6H₂O in a volume of approximately 45 milliliters of distilled water. The group B stock solution was autoclaved at 121° C. Group C included 97.5 milligrams thiamine-HCl, 1.6 milligrams vitamin B12, 33.3 milligrams pantothenic acid hemi-calcium salt, and 35.8 micrograms biotin dissolved in approximately 10 milliliters and filter sterilized. Group D included approximately 700 milliliters of sugar syrup obtained from Raceland Sugar in Louisiana. After the fermentor was cooled to 27° C., groups B, C, and D were added to the fermentor. Using sodium hydroxide and sulfuric acid, the fermentor was pH adjusted to 5 and the dissolved oxygen was spanned to 100 percent prior to inoculation. The fermentor volume prior to inoculation was approximately 4.85 liters.

The fermentor was inoculated with 0.75 liters of broth from the inoculum fermentation described above. The fermentation was pH controlled utilizing a 0.27 liter solution of 6N ammonium hydroxide at a pH of 5. The dissolved oxygen was controlled to maintain a target set point of 20 percent throughout the fermentation using agitation from 357 revolutions per minute to 1100 revolutions per minute, airflow from 0.9 liters per minute to 7.9 liters per minute, and oxygen from 0 liters per minute to 7 liters per minute. Throughout the fermentation, 4.9 liters of (Raceland) sugar syrup was fed to maintain a total sugar (glucose+fructose+sucrose) concentration less than 75 grams per liter. After 89 hours, the fermentor included 1104 grams of biomass that included 553 grams of fatty acids. The final cell density was 110.4 grams per liter dry weight. The fatty acid content was 50.1 percent of cellular dry weight, the average fatty acid productivity was 14.96 grams per liter per day and the resulting fatty acid yield (grams of fatty acids produced per grams of carbon feedstock) of the culture was 0.1634. The highest peak fatty acid productivity of the culture, measured over an 8 hour period was 31.9 grams per liter per day. The highest peak fatty acid productivity of the culture measured over a 24 hour period was 29.9 grams per liter per day. The fatty acid yield (grams of fatty acid produced per grams of carbon feedstock) at 27° C., pH 5 and 0.5× nitrogen was 0.1634.

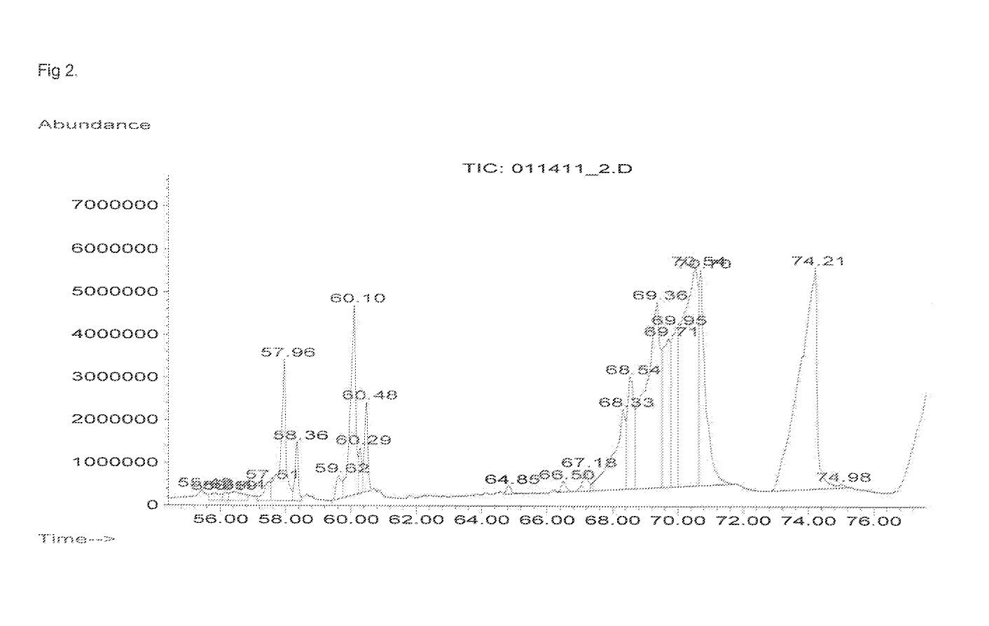

Analysis for sterol glycoside levels is carried out essentially according to the following procedure described above in connection with Example 1. Results of GC-MS analysis for the product of this example are shown in FIG. 2.

### Comparative Example

The green algae Chlorella protothecoides UTEX 250 (MK28415) was cultivated in a 14 liter New Brunswick Scientific BioFlo 3000 fermentor with a carbon (acid hydrolyzed sucrose syrup) and nitrogen (ammonium hydroxide) fed-batch process. The fermentation was inoculated with 1 liter of inoculum culture. For inoculum propagation a 14 liter Broadley James BioNet fermentor was utilized. The inoculum medium included 10 liters of medium prepared in five separate groups. Group A included 20 grams MSG*1H₂O, 2.9 grams CaCl₂*2H₂O, 10 grams yeast extract (T154), and 1.0 milliliters Dow 1520US (antifoam). Group A was autoclaved at 121 degrees in the inoculum fermentor at a volume of approximately 9.5 liters. Group B included 20 grams KH₂PO₄ in a volume of approximately 200 milliliters. The group B stock solution was autoclaved at 121° C. Group C included 103 milligrams FeSO₄*7H₂O, 184.4 milligrams citric acid, 18.1 milligrams MnCl₂*4H2O, 2.2 milligrams ZnSO₄*7H2O, 049 milligrams CoCl₂*6H₂O, 3.9 milligrams Na₂MoO₄*2H₂O, 28.6 milligrams H₃BO₃, and 0.79 milligrams CuSO₄*5H₂O all dissolved in distilled water. The group C stock solution was autoclaved at 121° C. Group D included 97.5 milligrams thiamine-HCl, 1.6 milligrams vitamin B12, and 33.3 milligrams pantothenic acid hemi-calcium salt dissolved in approximately 20 milliliters distilled water and filter sterilized. Group E included 1000 milliliters of distilled water containing 500 grams corn syrup. After the fermentor was cooled to 27° C., groups B, C, D, and E were added to the fermentor. Using sodium hydroxide and sulfuric acid, the fermentor was pH adjusted to 7 and the dissolved oxygen was spanned to 100 percent prior to inoculation.

The inoculum fermentor was inoculated with 400 milliliters of a standard shake flask culture and cultivated at 27° C., pH 7, 384 revolutions per minute agitation, and 5 liters per minute of air for a period of 43.5 hours, at which point 1 liter was harvested from the fermentor and transferred to the 14 liter fermentor. The 14 liter fermentor included 10 liters of fermentation media. The fermentation media was prepared in a similar fashion to the inoculum fermentor. The fermentation media included 5 batched media groups. Group A included 25 grams MSG*1H₂O, 2.9 grams CaCl₂*2H₂O, 10 grams yeast extract (T154), and 1.0 milliliters Dow 1520US (antifoam). Group A was autoclaved at 121° C. in the fermentor at a volume of approximately 5.5 liters. Group B included 25 grams KH₂PO₄ in a volume of approximately 250 milliliters. The group B stock solution was autoclaved at 121° C. Group C included 257.5 milligrams FeSO₄*7H₂O, 461 milligrams citric acid, 45.25 milligrams MnCl₂*4H2O, 5.55 milligrams ZnSO₄*7H2O, 1.225 milligrams CoCl₂*6H₂O, 9.75 milligrams Na₂MoO₄*2H₂O, 71.5 milligrams H₃BO₃, and 1.975 milligrams CuSO₄*5H₂O all dissolved in distilled water. The group C stock solution was autoclaved at 121° C. Group D included 35.8 micrograms biotin, 97.5 milligrams thiamine-HCl, 1.6 milligrams vitamin B12, and 33.3 milligrams pantothenic acid hemi-calcium salt dissolved in approximately 20 milliliters distilled water and filter sterilized. Group E included approximately 1000 milliliters of hydrolyzed sugar syrup obtained from Raceland Sugar in Louisiana. The sugar syrup was hydrolyzed by adding sulfuric acid to a pH of about 4 and sterilizing at 121° C. for at least one hour. After the fermentor was cooled to 27° C., groups B, C, D, and E were added to the fermentor. Using sodium hydroxide and sulfuric acid, the fermentor was pH adjusted to 7 and the dissolved oxygen was spanned to 100 percent prior to inoculation. The fermentor volume prior to inoculation was approximately 5.9 liters.

The fermentor was inoculated with 1 liter of broth from the inoculum fermentation described above. The fermentation was pH controlled utilizing a 0.27 liter solution of 6N ammonium hydroxide at a pH of 7 until the ammonium hydroxide feed was exhausted (approximately 55 hours after inoculation), at which point 4N sodium hydroxide was used for the remainder of the fermentation. The dissolved oxygen was controlled to maintain a target set point of 20 percent throughout the fermentation using agitation from 357 revolutions per minute to 850 revolutions per minute and airflow at 8 liters per minute. Throughout the fermentation, 5.2 liters of (Raceland) sugar syrup was fed to maintain a total sugar (glucose+fructose+sucrose) concentration less than 55 grams per liter. After 93 hours, the fermentor included 1082 grams of biomass that included 462.4 grams of fatty acids. The final cell density was 108.2 grams per liter dry weight. The fatty acid content was 42.73 percent of cellular dry weight, the average fatty acid productivity was 11.93 grams per liter per day and the resulting fatty acid yield (grams of fatty acids produced per grams of carbon feedstock) of the culture was 0.17617. The highest peak fatty acid productivity of the culture, measured over an 8 hour period was 33.6 grams per liter per day. The highest peak fatty acid productivity of the culture measured over a 24 hour period was 24.4 grams per liter per day. The fatty acid yield (grams of fatty acid produced per grams of carbon feedstock) at 27° C., pH 7 and 0.5× nitrogen was 0.17617.

It will be apparent to those skilled in the art that various modifications and variations can be made in the disclosed structures and methods without departing from the scope or spirit of the invention. Particularly, descriptions of any of the embodiments can be freely combined with descriptions of other embodiments to result in combinations and/or variations of two or more elements and/or limitations. Other embodiments of the invention will be apparent to those skilled in the art from consideration of the specification and practice of the invention disclosed herein. It is intended that the specification and examples be considered exemplary only, with a true scope and spirit of the invention being indicated by the following claims.

What is claimed is:

1. An oil suitable for the production of a biofuel, wherein the oil comprises:
substantially no sterol glycosides, and
material obtained from a culture comprising one or more oleaginous microorganisms.

2. The oil of claim 1, comprising less than about 15 parts per million of sterol glycosides by weight.

3. The oil of claim 1, wherein the material comprises at least one fatty acid triglyceride.

4. The oil of claim 1, wherein the culture comprises a bacteria, cyanobacteria, algae, or fungus.

5. The oil of claim 1, wherein the culture comprises Sporidiobolus pararoseus or Rhodotorula ingenosa.

6. The oil of claim 1, wherein the oil comprises at least one fatty acid triglyceride comprising a fatty portion having from about 8 carbon atoms to about 30 carbon atoms.

7. A biofuel made from the oil of claim 1.

8. The biofuel of claim 7, wherein the biofuel meets or exceeds biodiesel standard set forth in ASTM standard specification D6751-11b or the European specification EN14214.

9. The biofuel of claim 7, wherein the biofuel meets or exceeds low temperature flow test as set forth in ASTM standard specification D4539.

10. A biofuel, wherein the biofuel comprises:
substantially no sterol glycosides, and
at least one fatty acid C₁-C₄ alkyl ester derived from an oil obtained from a culture comprising one or more oleaginous microorganisms.

11. The biofuel of claim 10, wherein the biofuel comprises at least one fatty acid methyl ester (FAME).

12. A method of producing a feedstock suitable for use in the production of a biofuel, the method comprising:
growing a culture of an oleaginous microorganism in a presence of a carbon source,
wherein material produced by the culture comprises substantially no sterol glycosides.

13. The method of claim 12, wherein the culture comprises a bacteria, cyanobacteria, algae, or fungus.

14. The method of claim 12, wherein the culture comprises a yeast.

15. A biofuel produced by the method of claim 12.

16. A method according to claim 12, comprising no processing to reduce sterol glycoside levels.

17. A method of producing a fatty acid derivative composition, the method comprising:
growing a culture of an oleaginous microorganism in the presence of a carbon source, wherein material produced by the microorganism comprises substantially no sterol glycosides,
lysing the microorganism to produce a lysate; and
isolating an oil from the lysate.

18. A method according to claim 17, further comprising hydrolyzing the oil to produce a hydrolysis product comprising one or more acids, one or more acid salts, or combinations thereof.

19. A method according to claim 17, further comprising esterifying the hydrolysis product with an alcohol comprising about one to about four carbon atoms.

20. A fatty acid methyl ester composition comprising:
substantially no sterol glycosides, and
at least one fatty acid methyl ester derived from an oil obtained from a culture comprising one or more oleaginous microorganisms.

21. A fatty acid triglyceride composition comprising:
substantially no sterol glycosides, and
at least one fatty acid triglyceride obtained from a culture comprising one or more oleaginous microorganisms.

22. A production system comprising:
a fatty triglyceride production vessel adapted to receive a carbon source, and a culture of an oleaginous microorganism,
wherein material produced by the microorganism comprises substantially no sterol glycosides.
